(19) United States
(12) Patent Application Publication
(10) Pub. No.: US 2024/0325647 A1
(43) Pub. Date: Oct. 3, 2024

(54) Components and sub-assemblies for medicament delivery devices

(71) Applicant: SHL Medical AG, Zug (CH)

(72) Inventors: Meng-Jhen Chiou, Taoyuan City (TW); Tzu Wei Liu, Taoyuan City (TW)

(21) Appl. No.: 18/709,854

(22) PCT Filed: Nov. 3, 2022
(86) PCT No.: PCT/EP2022/080692

§ 371 Date: May 14, 2024

(30) Foreign Application Priority Data
Nov. 17, 2021 (EP) 21208876.9

(51) Int. Cl. A61M 5/28; A61M 5/20; A61M 5/31

(52) U.S. Cl. CPC A61M 5/281; A61M 5/20; A61M 2005/3143; A61M 2205/583

(57) ABSTRACT

The present disclosure provides a sub-assembly for a medicament delivery device. The sub-assembly includes a tubular rotator, a tubular housing, and a syringe support. The rotator includes a protrusion extending from an outer surface of the rotator. The syringe support includes a helically extending slot on an inner surface of the syringe support, the protrusion being in the helically extending slot. The syringe support includes an axially extending slot on an outer surface of the syringe support. The housing includes an axially extending rib extending from an inner surface of the housing, the axially extending rib being in the axially extending slot.

### TECHNICAL FIELD

The application concerns components and sub-assemblies for medicament delivery devices such as autoinjectors.

### BACKGROUND

Medicament delivery devices such as autoinjectors have already been very commercially successful due to a combination of properties such as robustness, simplicity and usability. Nevertheless, the applicant has appreciated that there is still further scope for improvement of some features to help prevent issues such as syringe flange breakage and rattling sounds in medicament delivery devices.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

A first aspect concerns a sub-assembly for a medicament delivery device, the sub-assembly comprising a tubular rotator, a tubular housing, and a syringe support, wherein the rotator comprises a protrusion extending from an outer surface of the rotator, wherein the syringe support comprises a helically extending slot on an inner surface of the syringe support, the protrusion being in the helically extending slot, wherein the syringe support comprises an axially extending slot on an outer surface of the syringe support, and wherein the housing comprises an axially extending rib extending from an inner surface of the housing, the axially extending rib being in the axially extending slot. This can help prevent syringe flange breakage, and can also help reduce or prevent rattling sounds within a device.

Optionally, the syringe support comprises a flexible structure at a proximal end of the syringe support. Optionally, the housing comprises a syringe shoulder support extending from an inner surface of the housing. Optionally, the housing comprises a window.

Another aspect concerns a medicament delivery device comprising a sub-assembly as described above. Optionally, the medicament delivery device is an autoinjector.

Another aspect concerns a plunger rod comprising a flexible arm for engaging a distal end of a syringe. Another aspect concerns one or more flexible materials on a rear housing of a medicament delivery device and/or on a housing of a medicament delivery device. Another aspect concerns a compensation spring assembly for a medicament delivery device.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a perspective view of a rotator.

FIG. 2 shows a perspective view of a syringe support.

FIG. 3 shows a perspective view of an alternative syringe support.

FIG. 4 shows a perspective view of part of a housing.

FIG. 5 shows a cross-section view of part of a medicament delivery device, the medicament delivery device comprising the components shown in FIGS. 1, 2 and 4.

FIG. 6 shows an autoinjector as an example medicament delivery device.

FIG. 7 shows a perspective view of part of a medicament delivery device.

FIG. 8 shows a cross-section view of part of the medicament delivery device of FIG. 7.

FIG. 9 shows a perspective view of part of a plunger rod.

FIG. 10 shows a perspective view of a plunger rod.

FIG. 11 shows a perspective view of a rear housing.

FIG. 12 shows a perspective view of a housing.

FIG. 13 shows a perspective view of the rear housing of FIG. 11 attached to the housing of FIG. 12.

FIG. 14 shows a perspective view of an alternative rear housing.

FIG. 15 shows a perspective view of the rear housing of FIG. 14 attached to the housing of FIG. 12.

FIG. 16 shows a perspective view of a compensation spring assembly.

FIG. 17 shows a perspective view of another compensation spring assembly.

FIG. 18 shows a perspective view of the components of the compensation spring assembly of FIG. 16.

FIG. 19 shows a perspective view of the components of the compensation spring assembly of FIG. 17.

FIG. 20 shows a perspective view of part of a medicament delivery device comprising the compensation spring assembly of FIG. 16.

FIG. 21 shows a perspective view of part of a medicament delivery device comprising the compensation spring assembly of FIG. 17.

### DETAILED DESCRIPTION

In general, the concepts described herein could be implemented in various medicament delivery devices, for example in autoinjectors. An example of a medicament delivery device that could be adapted to include one or more of the concepts described herein is described in WO 2011/123024, which is herein incorporated by reference. Briefly, a medicament delivery device would comprise a housing (such as an outer housing), a medicament delivery member guard and the rotator. The rotator is able to move rotationally (in the circumferential direction) within the housing during use of the medicament delivery device. The medicament delivery member guard is able to move in the axial direction within the housing during use of the medicament delivery device. Typically, axial movement of the rotator relative to the housing would be restricted. Typically, rotational movement of the medicament delivery member guard relative to the housing would be restricted. The medicament delivery device also typically comprises a powerpack inside the housing, the powerpack comprising the rotator and a plunger rod, a medicament barrel inside the outer housing and a cap. The medicament barrel typically comprises a medicament container and a medicament delivery member such as a needle. Alternatively, a different medicament delivery member, such as a jet injector, could be used.

FIG. 6 shows an example autoinjector 100 extending along an axis 101 in an axial direction 102 between a distal end 103 and a proximal end 104, with a radial direction 105 and a circumferential direction 106 also depicted for reference. The autoinjector 100 comprises a housing 110 with an optional window 112, an optional cap 130 and a rear housing 140.

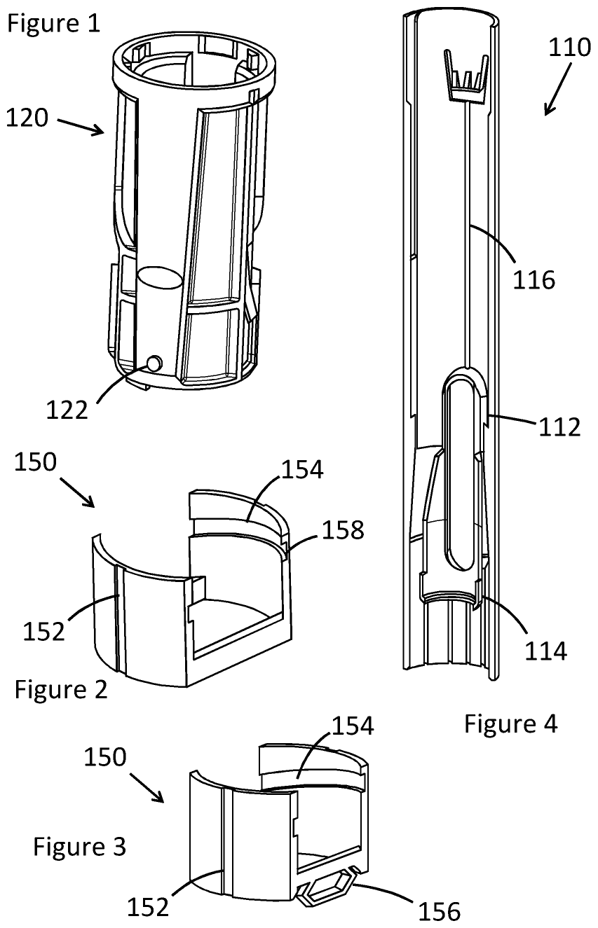

FIGS. 1 to 5 depict the components of an example sub-assembly. FIG. 1 shows a rotator 120, which comprises a protrusion 122. FIG. 4 shows part of a housing 110, which comprises a syringe shoulder support 114 and an axial rib 116. The axial rib 116 engages the axial slot 152. FIGS. 2 and 3 show two options for a syringe support 150. The syringe support 150 comprises an axial slot 152 and a helical slot 154. The helical slot 154 engages the protrusion 122. In the syringe support 150 in FIG. 3, a flexible structure 156 is provided; the flexible structure 156 abuts a distal end of the syringe (not shown).

The flexible structure 156 is an integral part of the syringe support 150 in the example in FIG. 3, but could alternatively or additionally comprise a flexible material attached to the proximal end of the syringe support 150 (e.g. a TPE (thermoplastic elastomer), which could be added by overmoulding, or rubber). Alternatively or additionally, the flexible material could also be added to a proximal end edge 158 of the helical slot 154. Provision of flexible material can help by allowing greater tolerance margins in component manufacture and/or can help with reducing the forces transmitted during medicament delivery device use, which is particularly relevant in reducing the forces transmitted to the syringe so as to avoid syringe breakage.

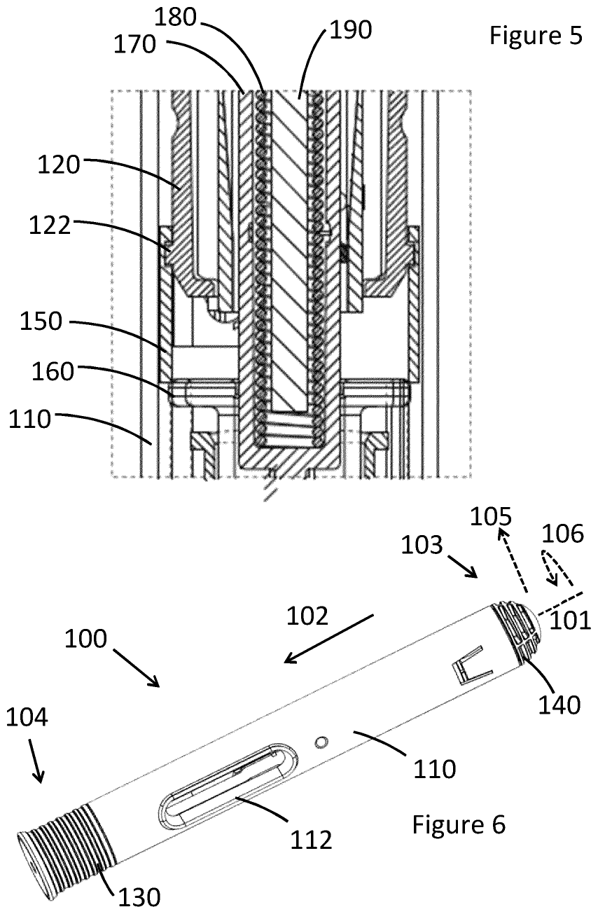

In a completed medicament delivery device before activation of the device, the proximal end of the syringe support 150 (e.g. the flexible structure 156) abuts the syringe 160, as can be seen in FIG. 5. When the rotator 120 is rotated (relative to the housing 110) during device activation, the syringe support 150 will be moved in the distal direction relative to the housing 110 as the protrusion 122 follows the helical slot 154. The interaction between the axial rib 116 and the axial slot 152 stops the syringe support 150 from rotating with the rotator. As a result, the syringe support 150 is moved away from the syringe 160 and no longer supports the syringe during medicament delivery. During medicament delivery, distal movement of the syringe relative to the housing is instead restricted by the plunger rod pushing against a stopper (not shown) of the syringe.

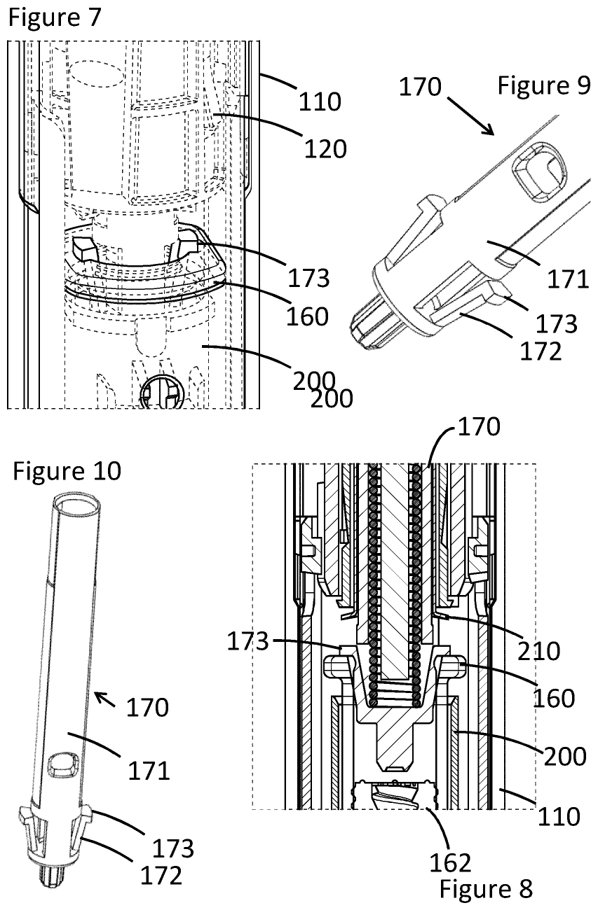

FIGS. 7 to 10 depict a second example. In particular, FIGS. 9 and 10 show an example plunger rod 170 that could be used to support the syringe instead of or in addition to the syringe support 150 described above. The plunger rod comprises a body 171 (which in this case is tubular) and a flexible arm 172. The proximal end of the arm 172 is attached to the body 171, and the distal end of the arm can then flex relative to the body 171. At the distal end of the arm, a protrusion 173 extends away from the arm 172. As shown in FIG. 7, a proximal face of the protrusion 173 engages a distal face of the syringe 160. Various medicament delivery device components (with many parts of them shown as dotted lines in FIG. 7 to enable clearer viewing of some of the most relevant parts) are also shown for context in FIGS. 7 and 8, including a housing 110, a rotator 120, a syringe 160 (including a stopper 162), a syringe carrier 200 and an optional U-bracket 210. The syringe carrier could be an integral part of the housing. The U-bracket could provide an end click at the end of medicament delivery.

During use of a medicament delivery device comprising the plunger rod of FIGS. 7 to 10, upon activation of the device (for example by a spring or by a powerpack with another power source, for example a gas or electric-powered powerpack), the plunger rod would be pushed in the proximal direction relative to the syringe. As a result, the distal ends of the arms would be pushed inwards by the proximally directed force, pushing the protrusions towards the axis and thereby allowing the protrusions to enter inside the syringe. As with the previous example in FIGS. 1 to 5, the arms therefore support the syringe prior to device use, and during device use the plunger rod (specifically the proximal end of the plunger rod, where the plunger rod abuts the stopper) supports the syringe (and can thereby stop the syringe from rattling).

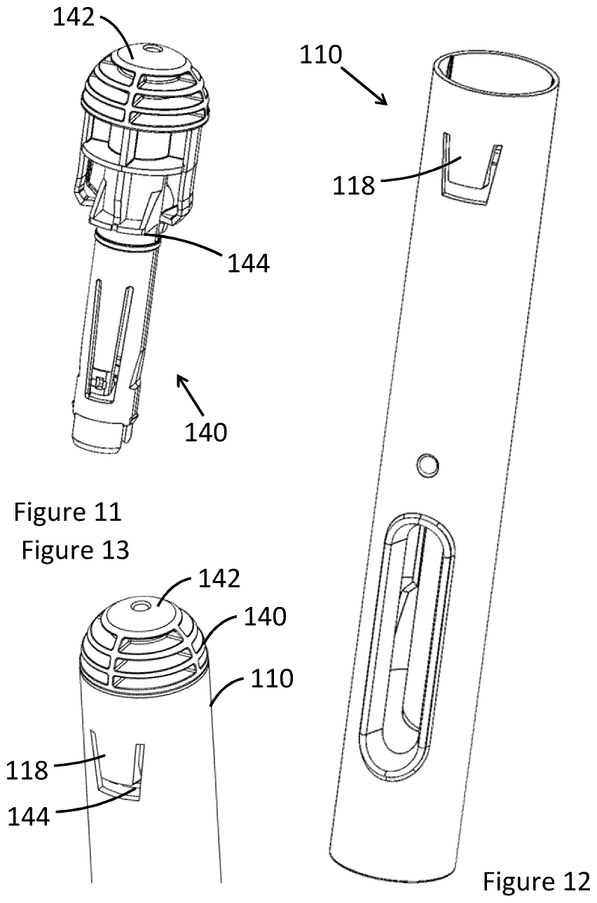

FIGS. 11 to 13 are provided to support the description of another example; this example could be used in addition to or instead of the examples described above. In this example, flexible material (e.g. a TPE (thermoplastic elastomer), which could be added by overmoulding, or rubber) is added in one or more locations on the rear housing 140 and/or one or more locations on the housing 110.

A first location is a distal end area 142 of the rear housing 140. A flexible material at this location can reduce the force transmitted to the syringe when a medicament delivery device comprising the rear housing is dropped with this area of the medicament delivery device being the area impacted (for example if the medicament delivery device is dropped on the floor with this area of the device hitting the floor).

Further locations where a flexible material could be used include on a clip 144 of the rear housing 140 and/or on an arm 118 of the housing 110. The clip 144 engages the arm 118. To reduce force transmission (for example when a medicament delivery device comprising the rear housing 140 and the housing 110 is dropped with the rear housing 140 impacting an object such as a floor), part or all of one or more of the clip 144 and the arm 118 could comprise a flexible material (alternatively or additionally, flexible material could be added to the existing structures as shown in FIGS. 11 and 12).

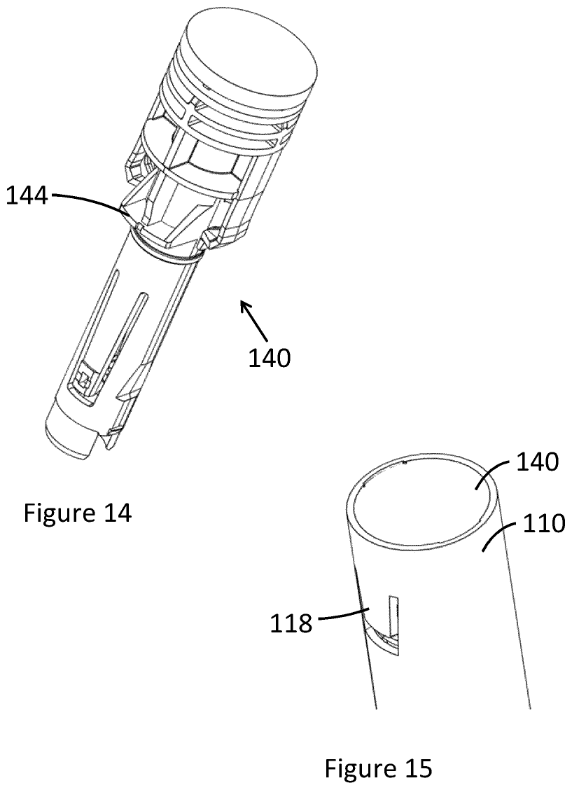

An alternative approach is shown in FIGS. 14 and 15. Instead of the distal end of the rear housing 140 extending further in the distal direction than the housing 110, the distal end of the rear housing 140 is inside the housing 110, as can be seen in FIG. 15. As a result, if the device is dropped, the housing 110 is typically the first thing to impact an object such as a floor. This allows a flexible material on one or more of the clip 144 and the arm 118 to cushion an impact.

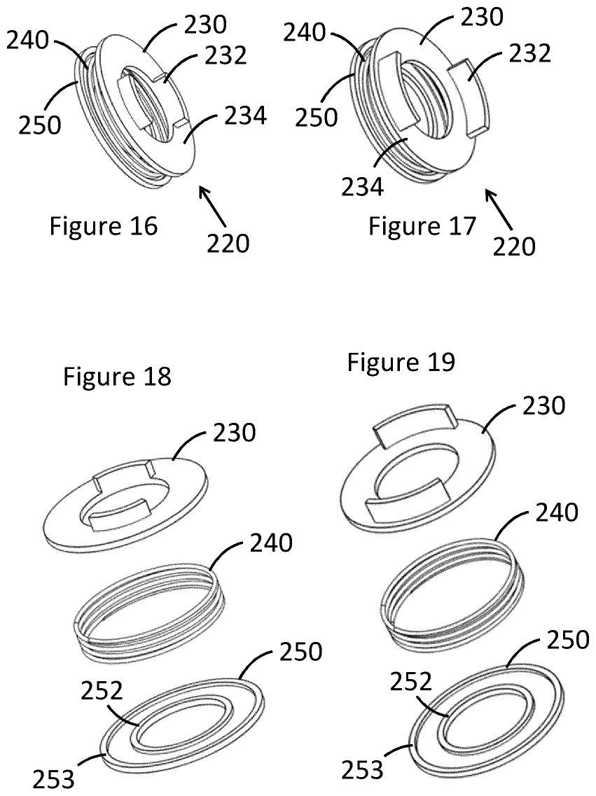

FIG. 16 shows an example compensation spring assembly 220. As can be seen more clearly in FIG. 18, the compensation spring assembly 220 comprises three components, namely a distal plate 230, a proximal plate 250 and a spring 240 arranged between the distal plate 230 and the proximal plate 250. The distal plate 230 comprises two distally extending protrusions 232 extending from a disc 234 (though one, three or more could be provided instead, as is generally the case with duplicated parts in medicament delivery devices such as those described herein). The distally extending protrusions 232 are optional, but can be beneficial, for example to leave space for part of a U-bracket between the rotator and the disc of the distal plate 230 (without distally extending protrusions, the distal face of the disc 234 could directly abut a proximally facing surface of a component such as a rotator or rear housing) (in an embodiment, similar proximally extending protrusions could extend from the proximal face of the disc of the proximal plate to engage a distally facing surface of a component such as a syringe). The proximal plate is also a disc 251, and has optional ribs 252, 253 extending in the distal direction from the distal surface of the disc; these ribs can help hold the spring 240 in place. Similar ribs (not shown) are optionally provided extending in the proximal direction from the proximal surface of the disc 234; these ribs can also help hold the spring 240 in place.

A compensation spring assembly like this can be used to help absorb shocks, for example from dropping a device. It can also help with tolerance management within a device; for example, the compensation spring assembly can allow for larger tolerances and/or can reduce or stop a device from rattling.

FIG. 20 shows the compensation spring assembly 220 of FIG. 16 with part of a medicament delivery device for context. In particular, a rotator 120, a rear housing 140, a syringe 160, a syringe carrier 200 and a medicament delivery member guard 260 (e.g. a needle guard) are shown in addition to the distal plate 230, the spring 240 and the proximal plate 250 of the compensation spring assembly. The proximal surface of the proximal plate 250 abuts a distal surface of the distal end of the syringe 160. The distal end of the distally extending protrusion 232 abuts a proximal surface of the rear housing 140. When in a medicament delivery device, the spring 240 would typically be slightly compressed.

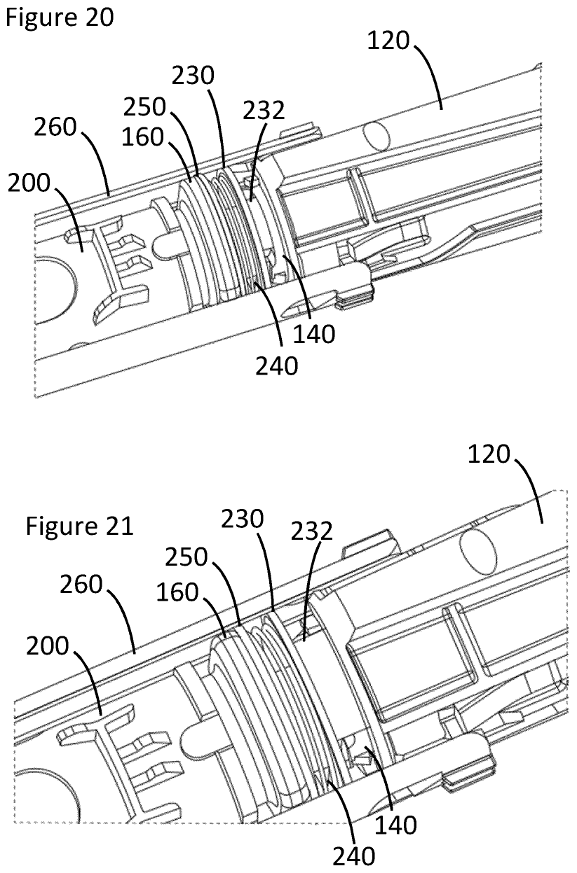

FIGS. 17, 19 and 21 show an alternative compensation spring assembly 220 in which the distally extending protrusions 232 are arranged in a different location on the distal face of the disc 234 to abut a different component (the rotator 120 instead of the rear housing 140).

Various modifications to the embodiments described are possible and will occur to those skilled in the art without departing from the invention which is defined by the following claims.

1-6. (canceled)

7. A sub-assembly for a medicament delivery device, the sub-assembly comprising:
a tubular rotator;
a tubular housing; and
a syringe support,
wherein the rotator comprises a protrusion extending from an outer surface of the rotator,
wherein the syringe support comprises a helically extending slot on an inner surface of the syringe support, the protrusion being in the helically extending slot,
wherein the syringe support comprises an axially extending slot on an outer surface of the syringe support,
and wherein the housing comprises an axially extending rib extending from an inner surface of the housing, the axially extending rib being in the axially extending slot.

8. The sub-assembly of claim 7, wherein the syringe support comprises a flexible structure at a proximal end of the syringe support.

9. The sub-assembly of claim 7, wherein the housing comprises a syringe shoulder support extending from an inner surface of the housing.

10. The sub-assembly of claim 7, wherein the housing comprises a window.

11. A medicament delivery device comprising the sub-assembly of any of claim 7.

12. The medicament delivery device of claim 11, wherein the medicament delivery device is an autoinjector.
